(19) United States
(12) Patent Application Publication
(10) Pub. No.: US 2004/0225222 A1
(43) Pub. Date: Nov. 11, 2004

(54) Real-time contemporaneous multimodal imaging and spectroscopy uses thereof

(76) Inventors: Haishan Zeng, Vancouver (CA); Mirjan Petek, Richmond (CA); Branko Palcic, Vancouver (CA); Gary Ferguson, Burnaby (CA)

Correspondence Address: TREXLER, BUSHNELL, GIANGIORGI,, BLACKSTONE & MARR, LTD., 105 WEST ADAMS STREET, SUITE 3600, CHICAGO, IL, 60603, US

(21) Appl. No.: 10/431,939

(22) Filed: May 8, 2003

(51) Int. Cl. A61B 6/00

(52) U.S. Cl. 600/476

(57) ABSTRACT

The present invention comprises an optical apparatus, methods and uses for real-time (video-rate) multimodal imaging, for example, contemporaneous measurement of white light reflectance, native tissue autofluorescence and near infrared images with an endoscope. These principles may be applied to various optical apparati such as microscopes, endoscopes, telescopes, cameras etc. to view or analyze the interaction of light with objects such as planets, plants, rocks, animals, cells, tissue, proteins, DNA, semiconductors, etc. Multi-band spectral images may provide morphological data such as surface structure of lung tissue whereas chemical make-up, sub-structure and other object characteristics may be deduced from spectral signals related to reflectance or light radiated (emitted) from the object such as luminescence or fluorescence, indicating endogenous chemicals or exogenous substances such as dyes employed to enhance visualization, drugs, therapeutics or other agents. Accordingly, one embodiment of the present invention discusses simultaneous white light reflectance and fluorescence imaging. Another embodiment describes the addition of another reflectance imaging modality (in the near-IR spectrum). Input (illumination) spectrum, optical modulation, optical processing, object interaction, output spectrum, detector configurations, synchronization, image processing and display are discussed for various applications.

### FIELD OF INVENTION

Various optical apparati such as microscopes, endoscopes, telescopes, cameras etc. support viewing or analyzing the interaction of light with objects such as planets, plants, rocks, animals, cells, tissue, proteins, DNA, semiconductors, etc. Some multi-band spectral images provide morphological image data whereas other multi-band spectral images provide information related to the chemical make-up, sub-structure and/or other target object characteristics which may be measured from multi-band spectral images of reflected or emitted light. These light emission images, such as luminescence or fluorescence, may indicate and provide means to assess endogenous chemicals or exogenous substances such as dyes employed to enhance visualization, drugs, therapeutic intermediaries, or other agents.

In the field of medical imaging and more particularly endoscopy, reflected white light, native tissue autofluorescence, luminescence, chemical emissions, near-IR reflectance, and other spectra provide a means to visualize tissue and gather diagnostic information. In addition to visualization of tissue morphology the interaction of light in various parts of the electromagnetic spectrum has been used to collect chemical information. Three general real-time imaging modalities for endoscopy that are of interest include white-light reflectance imaging, fluorescence emission and near infrared reflectance imaging modalities.

In endoscopy, conventional white light imaging is typically used to view surface morphology, establish landmarks, and assess the internal organs based on appearance. Applications for viewing the respiratory and gastro-intestinal tracts are well established. Fluorescence imaging has evolved more recently and using the properties of tissue autofluorescence has been applied to the detection of early cancer. Similarly, observations of various native and induced chemical interactions, such as labeling tissue with proteins, for example, have been accomplished using fluorescence imaging. Near infrared light may be used to measure tissue oxygenation and hypoxia in healthy and diseased tissue. Alternatively, fluorescently-tagged monoclonal antibodies may be used to label specific cellular proteins, which in turn may be detected and/or be measured optically.

Presently, methods and device configurations exist which use each of these imaging modalities to gather data in real-time, at video-rate. However, for imaging, this real-time information from different modalities has been available sequentially or in part, but not simultaneously.

As used herein, “multimodal” means at least two imaging modes which differ in their spectral bands of illumination or their spectral bands of detection, or both.

“Optical modulator” as used herein means a device or combination of optical and/or electro-optical devices to alter the wavelength(s), and/or to alter the intensity, and/or to time-gate various spectra of electromagnetic radiation. Various filters, filter wheels, lenses, mirrors, micro-mirror arrays, liquid crystals, or other devices under mechanical or electrical control may be employed alone or in combination to comprise such an optical modulator. Certain embodiments of the present invention utilize two optical modulators, one associated with modulating light source spectrum that will be used to interrogate or interact with an object. Modulation of source illumination therefore could be as simple as switching (gating on) one or more illumination sources in a controlled manner, or accomplishing optical modulation with the devices as described. A second modulator is used to process the light returned after interacting with the object. The second optical modulator could be serve to split imaging light segments to direct them to various detectors, and be comprised of, for example a moving mirror, a rotating mirror as part of a filter wheel, or a digital multi-mirror device (DMD). The detectors may be imaging devices such as cameras with CCD sensors or these sensors may comprise spectrometers. In some cases, such as in vivo endoscopic use, interaction of source illumination may be with lung tissue and returned light may include various reflected and re-emitted spectra.

Control and synchronization as used herein means to provide control over the optical modulators and/or the electromagnetic radiation source and/or the detectors, for example at real-time video rates, and to further synchronize the operation of these components to provide a means to generate the desired source spectrum for the desired time periods, and to process (e.g. amplify, attenuate, divide, gate) and detect image signals of various spectrum, contemporaneously. In some embodiments relatively tight control and synchronization are required, in other embodiments, these returned signals may themselves be used for co-ordination, for example, their intensity or wavelength may be used to provide information for control and synchronization.

In addition to viewing and analysis, at the same time, selected spectra of light may be directed to stimulate certain photosensitive chemicals so that treatments such as photodynamic therapy (PDT) may be delivered and monitored.

While prior art discusses means to sequentially provide white-light imaging (typical spectral range 400 nm to 700 nm), fluorescence imaging (e.g. tissue autofluorescence stimulated with blue light from 400 nm to 450 nm and re-emitted in the 470 nm to 700 nm range) and near-infrared images with an approximate spectral range of 700 nm to 800 nm or beyond, and/or particular spectra in these ranges, and/or an imaging modality combined with a spectral signal, there remains a need for apparatus and methods to provide these various imaging modes, contemporaneously, at video rates. The present invention meets this need.

### BRIEF DISCUSSION OF ART

U.S. Pat. No. 6,364,829, to Fulghum, entitled, “Autofluorescence imaging system for endoscopy”, discusses a broad-band light source to provide both visible light (which induces minimal autofluorescence) and ultraviolet light (capable of inducing tissue autofluorescence). Images are detected, for example, by a single imaging detector at the distal tip of an endoscope and provisions are made for electronically switching between these source illumination spectrum. Various light sources, filter wheels, shutters, mirrors, dichroic mirrors, spectrum, light sources, intensities and timing diagrams are provided and therefore this prior art is included by reference.

U.S. Pat. No. 6,148,227, to Wagnieres, entitled, “Diagnosis apparatus for the picture providing recording of fluorescing biological tissue regions”, discusses illumination spectrum and components for fluorescence imaging. In one embodiment red and green components are directed to separate portions of a CCD with independent signal processing.

U.S. Pat. No. 6,061,591, to Freitag, entitled, “Arrangement and method for diagnosing malignant tissue by fluorescence observation”, discusses a strobed white-light illumination source and laser to stimulate fluorescence. Alternatively, a desired fluorescence spectrum may be isolated and provided from a single lamp, for example, a Mercury-Xenon arc lamp. Filter wheels (with red, green and blue filters as well as filters to divide fluorescence into red and green components) and timing requirements are also discussed. Measurements of white-light images and fluorescence are performed in sequence, although both may be displayed on the monitor. Various Figures describe light sources which are similar to those contemplated for the present invention.

The system described in Fulghum has the ability to switch back and forth between white light and fluorescence visualization methods electronically with display rates up to 10 Hz, or higher. Unlike other prior art (e.g. U.S. Pat. No. 5,647,368 which will be discussed), switching between normal visible light imaging, in full color, and fluorescence imaging is accomplished by an electronic switch rather than by physical modulation (switching) by the operator. This prior art also discusses a fluorescence excitation light at ultraviolet to deep violet wavelengths placed at the end of an endoscope, as well as gallium nitride laser diodes and mercury arc lamps for UV which are also contemplated as illumination sources for various embodiments of the present invention. Also of interest, Fulghum discusses limitations of endoscopes and more particularly limitations related to the UV-transmissive properties of optical fibers. Some of these limitations are addressed by co-pending U.S. application Ser. No. 10/226,406 to Ferguson/Zeng, filed approximately Aug. 23, 2002, entitled “Non-coherent fiber optic apparatus and imaging methods”.

U.S. Pat. No. 6,019,719, to Schulz, entitled, “Fully auotclavable electronic endoscope”, discusses an objective lens, crystal filter, IR filter and CCD chip arranged at the distal end of an endoscope for imaging.

U.S. Pat. No. 5,930,424 to Heimberger, entitled, “Device for connecting a fiber optic cable to the fiber optic connection of an endoscope”, discusses various aspects of coupling devices such as light sources to an endoscope.

U.S. Pat. No. 5,926,213 to Hafele, entitled, “Device for correcting the tone of color pictures recorded by a video camera”, such as an endoscope camera, is discussed along with a rotary transducer to activate tone correction. Color correction, calibration or normalization is useful for quantization from image data or comparison of images and is considered for various embodiments of the present invention.

U.S. Pat. No. 5,827,190, to Palcic, entitled, “Endoscope having an integrated CCD sensor”, discusses illumination light sources and sensors to measure various signals associated with tissue and tissue disease.

U.S. Pat. No. 5,647,368, to Zeng, entitled, “Imaging system for detecting diseased tissue using native fluorescence in the gastrointestinal and respiratory tract”, among other things discusses use of a mercury arc lamp to provide for white light and fluorescence imaging with an endoscope to detect and differentiate effects in abnormal or diseased tissue.

U.S. Pat. No. 5,590,660, to MacAulay, entitled, “Apparatus and methodfor imaging diseased tissue using integrated autofluorescence” discusses light source requirements, optical sensors, and means to provide a background image to normalize the autofluorescence image, for uses such as imaging diseased tissue.

U.S. Pat. No. 5,769,792, to Palcic, entitled, “Endoscopic imaging system for diseased tissue”, further discusses light sources and means to extract information from the spectral intensity bands of autofluorescence, which differ in normal and diseased tissue.

Also co-pending U.S. patent application Ser. No. 09/741,731, to Zeng, filed approximately Dec. 19, 2000 and entitled, “Methods and apparatus for fluorescence and reflectance imaging and spectroscopy and for contemporaneous measurements of electromagnetic radiation with multiple measuring devices”, (a continuation-in-part of U.S. Publication No. 2002/0103439) discusses contemporaneous methods of providing one mode of imaging and spectroscopy contemporaneously, but multiple imaging and associated spectroscopy modalities is sequential.

In the present invention, methods are described to perform multimodal imaging contemporaneously at various desired wavelengths. Unlike Zeng's prior art, Zeng's present invention does not seek to provide images and measurements of wavelength spectrum, instead it seeks to provide contemporaneous multimodal imaging, where entire images in defined spectrum are detected and utilized for display or analysis.

U.S. Pat. No. 5,999,844, to Gombrich, entitled, “Method and apparatus for imaging and sampling diseased tissue using autofluorescence”, discusses a plurality of image detectors that receive excitation light as well as depositing biopsies in separate compartments or captive units.

U.S. Pat. No. 6,212,425, to Irion, entitled, “Apparatus for photodynamic diagnosis”, discusses endoscopic imaging using a light-induced reaction or intrinsic fluorescence to detect diseased tissue and delivery light for therapeutic use or to stimulate compounds that in turn provide therapy, for example.

U.S. Pat. No. 4,884,133, to Kanno, entitled “Endoscope light source apparatus”, discusses light sources, light guides and control of these elements for endoscopic use.

U.S. Pat. No. 5,749,830 to Kaneko entitled “Fluorescent endoscope apparatus” discusses use of two light sources, a first (e.g. lamp) for white light and a second (e.g. helium-cadmium laser) for fluorescence to provide interrogating spectrum. Kaneko '830 also employs a filter wheel placed in the pathway of a single detector. For multimodal imaging the filter wheel has a plurality of filters (e.g. three in FIGS. 4a and 5 in FIG. 4b). While they illustrate the display of two imaging modalities (110 of FIG. 7.), they do not discuss simultaneous real-time multimodal imaging. As this prior art discusses a wide range of issues utilized within the present invention, such as combining light sources, synchronization and filter wheels, '830 is included by reference herein.

Endoscopes and imaging applications are further discussed in co-pending U.S. application Ser. No. 10/226,406 to Ferguson/Zeng, entitled “Non-coherent fiber optic apparatus and imaging methods”, which among other things, discusses apparatus to overcome some existing limitations of fiber optic devices, such as endoscopes.

### SUMMARY AND OBJECTIVES OF THE INVENTION

The present invention solves the problems described above by providing simultaneous multimodal spectral images of a target object. Targeting radiation or illumination is modulated to provide segments of radiation of different wavelengths, for example, alternating segments of white, green, blue, red, and near-infrared light. The target object returns reflected and re-emitted (for example, fluoresced) light, which is further modulated to separate the returned light into segments corresponding to different wavelengths. The returned radiation can be processed, displayed, and analyzed.

### BRIEF DISCUSSION OF DRAWINGS

FIG. 1 (prior art) shows a series of typical desired spectra utilized for endoscopic imaging.

FIGS. 2a and 2b (prior art) illustrate the spectra from a typical fluorescence endoscopy system.

FIG. 3 (prior art) illustrates a typical spectra from the fluorescence mode of a sequential white light and fluorescence endoscopy system.

FIG. 4 shows an illumination source placed for example at the distal end of an endoscope FIG. 5 is a perspective view of an embodiment of the present invention FIG. 6a is a perspective view of the simultaneous white light and fluorescence imaging with a single detector comprising multiple sensors.

FIG. 6b is a perspective view of the detector configuration associated with FIG. 6a.

FIG. 6c is a perspective view of another detector configuration associated with FIG. 6a, which can be placed at the distal tip of an endoscope.

FIG. 6d is a block diagram of the control and synchronization for contemporaneous imaging modes described in FIGS. 6a, 6b and 6c.

### DETAILED DISCUSSION OF DRAWINGS AND PREFERRED EMBODIMENTS

While the invention may be susceptible to embodiments in different forms, there is shown in the drawings, and herein will be described in detail, specific embodiments with the understanding that the present disclosure is to be considered an exemplification of the principles of the invention, and is not intended to limit the invention to that as illustrated and described herein.

Endoscopy and endoscopic apparatus may be described and differentiated in terms of tissue illumination and generated signals which include reflected light and/or emission spectrum.

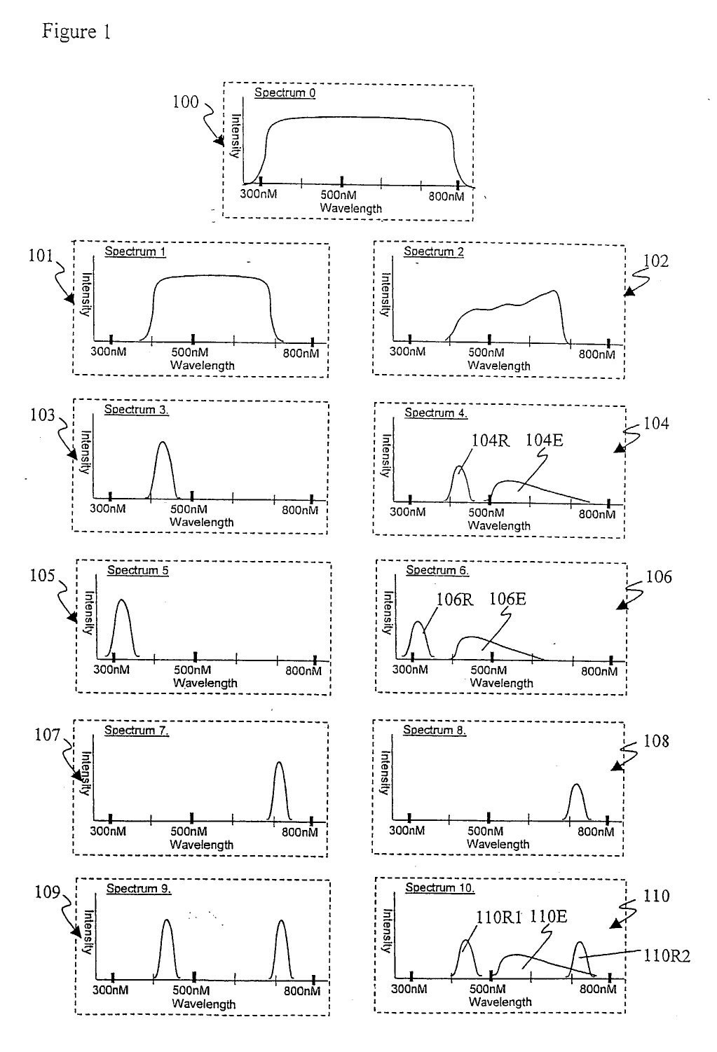

FIG. 1 (prior art) illustrates typical spectra utilized for white light and fluorescence assessment. Spectrum 0 100 shows the broad range of illumination typically utilized. Such illumination may be provided by a single source or multiple combined sources as discussed in prior art and further in this application.

Spectrum 1 101 shows a typical white light (broad-band) illumination spectrum. Various illumination sources (lamps etc.) are available to produce broad-band illumination, for example U.S. Pat. No. 6,364,829 to Fulghum discusses desired illumination. Illumination as shown in

Spectrum 1 101 may interact with a target tissue providing reflected light, such as typical white light signal (reflectance), illustrated in Spectrum 2 102, in substantially the same spectral range as the source, but attenuated relative to the incident illumination. Such attenuation may be preferential based on tissue absorption, presence of blood and other factors as observed in Spectrum 2 102.

Spectrum 3 103 represents typical short wavelength light, for example, blue light, intended to excite tissue fluorescence. A typical returned signal Spectrum 4 104 has two components, a tissue reflectance component 104R, which is typically not utilized, and a tissue fluorescence emission signal 104E. The reflectance component is often blocked or filtered out so that it does not interfere with fluorescence detection.

Accordingly, to excite tissue fluorescence, narrow illumination bands may be preferred. The narrow bands may be isolated from broad-band illumination or they may be provided by a narrow band source such as an LED or laser. Typical UV illumination as illustrated in Spectrum 5 105, may be used to excite tissue autofluorescence producing a spectrum such as is shown in Spectrum 6 106. Again, the reflectance component 106R is usually not used. Typical illumination illustrated in Spectrum 7 107 in the red/near IR provides a reflectance component as shown in Spectrum 8 108.

In addition, illumination spectrum may be combined and used to advantage. For example, typical illumination shown in Spectrum 9 109, blue light plus red/near IR light, produces a signal spectrum such as shown in Spectrum 10 110. These spectra (0 to 10) will be referred to during the discussion of various Figures.

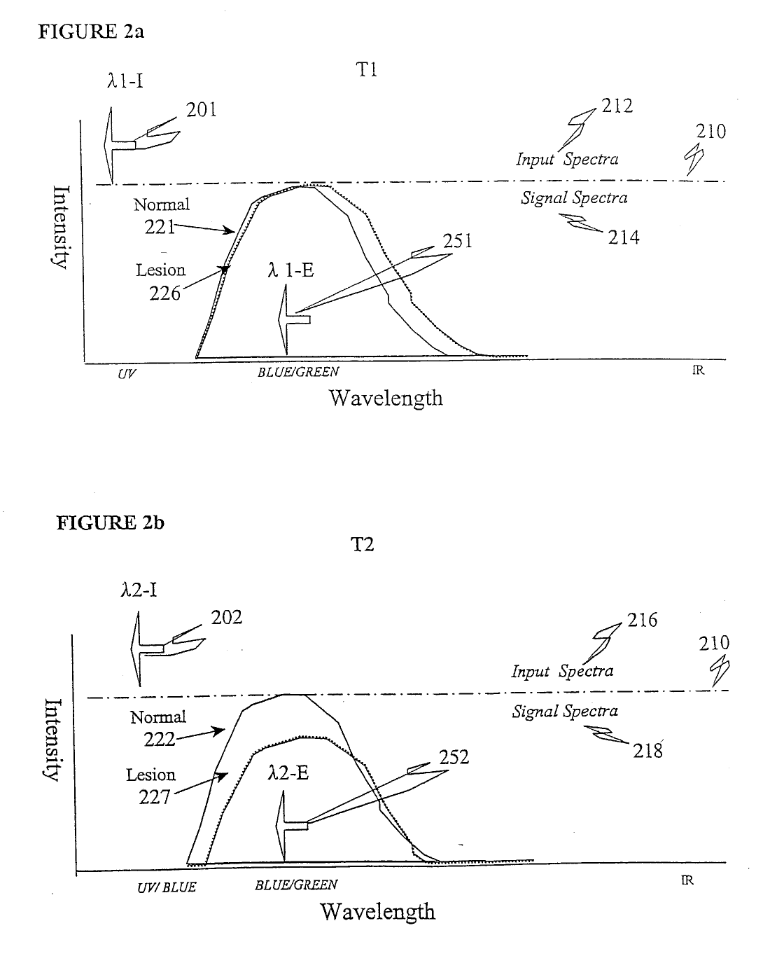

FIGS. 2a and 2b (prior art) describe and represent endoscopic imaging principles encompassing U.S. Pat. No. 5,413,108 to Alfano entitled, “Method and apparatus for mapping a tissue sample for and distinguishing different regions thereof based on luminescence measurements of cancer-indicative native fluorophor” and U.S. Pat. No. 6,091,985 to Alfano, entitled, “Detection of cancer and precancerous conditions in tissues and/or cells using native fluorescence excitation spectroscopy”, both of which are included herein by reference. As was introduced, these principals may be applied to other optical systems such as microscopes, cameras, telescopes etc. and are described in U.S. Pat. No. 6,080,584 to Alfano, entitled “Method and apparatus for detecting the presence of cancerous and precancerous cells in a smear using native fluorescence spectroscopy.” This prior art to Alfano is included by reference.

Accordingly, FIG. 2a illustrates white light, reflectance and emission endoscopy, generically, in terms of input spectra 212 (illumination) and output signal spectra 214, with input and output delineated by indicator line 210. A first illumination 201, λ1-I, is selected in the UV range to stimulate tissue autofluorescence (e.g. Spectrum 5 as discussed in association with FIG. 1). The resulting tissue emission spectra 251 occur in the blue/green region, which is further identified as λ1-E (e.g. 106E of Spectrum 6 in FIG. 1). Using the interrogating illumination 201, the emission signal intensities of normal and diseased tissue are similar. This is further shown by the characteristic curve for normal tissue 221 and diseased tissue, 226. A first representative (reference) image of tissue emission (autofluorescence) is typically acquired during time interval T1.

FIG. 2b shows input spectra 216 and signal spectra 218. During time interval T2, a second interrogating illumination 202, λ2-I in the UV/blue region, illuminates tissue to excite autofluorescence (e.g. Spectrum 3 discussed in association with FIG. 1). The resulting tissue emission spectra 252, further identified as λ2-E (emission) again occurs in blue/green region. Under these conditions, a measurable difference is observed between the characteristic curves for normal tissue 222 and diseased tissue 227. A tissue image is acquired during this interval, T2. Ratios and/or differences between the first (reference) image acquired during T1 and a second image acquired during T2 provides a basis to normalize, process and extract diagnostic information. One advantage of such a configuration is that, since the images are acquired sequentially, this may be accomplished using a single image sensor. Additionally, because the two tissue autofluorescence images are produced in the same general spectral region (251, 252 are both blue/green), they cannot be separated in space by optical means and are therefore separated in time domain (T1 and T2) as indicated. Various limitations result, for example, it becomes more difficult to register (pixel align) the two images which may be shifted due to breathing or motion of the organ or target tissue (e.g. lung).

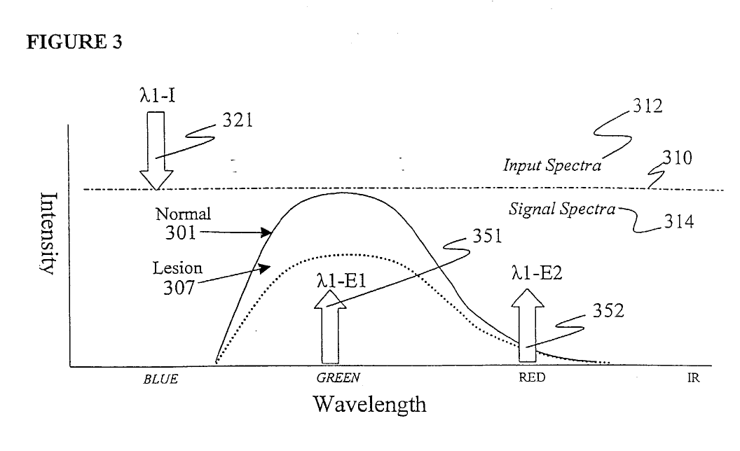

FIG. 3 (prior art) illustrates the fluorescence mode used for sequential white light and fluorescence endoscopy as discussed in U.S. Pat. No. 5,647,368, to Zeng, entitled “Imaging system for detecting diseased tissue using native fluorescence in the gastrointestinal and respiratory tract” and further discussed in U.S. Pat. No. 6,462,770 to Cline entitled, “Imaging system with automatic gain control for reflectance and fluorescence endoscopy”. As will be further described, Zeng '368 typically employs two illumination sources to provide sequential illumination spectra such as Spectrum 1 and Spectrum 3 as discussed in association with FIG. 1.

FIG. 3 shows input spectra 312 above line 310 and output spectra 314 below line 310 for the fluorescence imaging mode. An input spectra 321, further labeled λ1-I provides blue light such as Spectrum 3 discussed with FIG. 1 to excite tissue fluorescence. Tissue emission 351, further identified as λ1-E, occurs in the green region and typical tissue characteristic curves for normal tissue 301 and diseased tissue 307 are also indicated. In Zeng '368 optical modulation is accomplished, for example by turning off a broad-band white light source and turning on the blue light source as described above. And as will be described with FIG. 5 for the present invention, a second form of optical modulation is provided by inserting or displacing a mirror that directs either white light reflectance or fluorescence emissions to the desired detector(s). Accordingly, it is one objective of the present invention to provide a means to switch illumination spectra at video-rates, and coordinate the direction and capture of images. While it may be possible to physically accomplish this switching at a high rate, maintaining this switching, reproducibly, over an extended period is beyond the scope of the prior art, and is required to accomplish multimodal contemporaneous imaging as contemplated herein. These principals are further described in Cline '770 with FIG. 1 illustrating a combined light source (36) modulated by switching mode 106 and operator control switches 65. As this prior art also discusses, among other things, desired illumination it is included by reference.

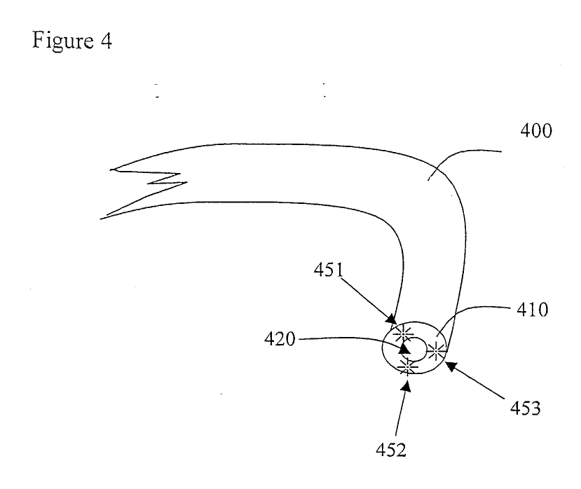

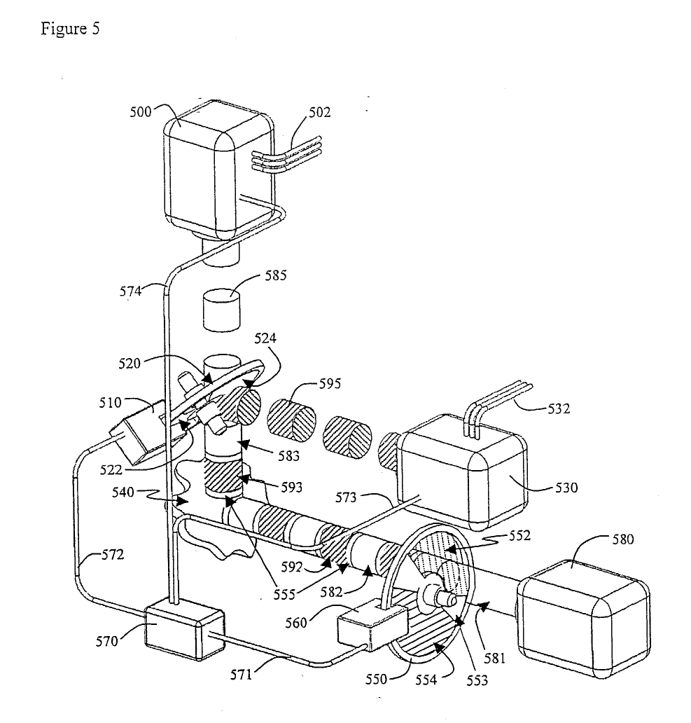

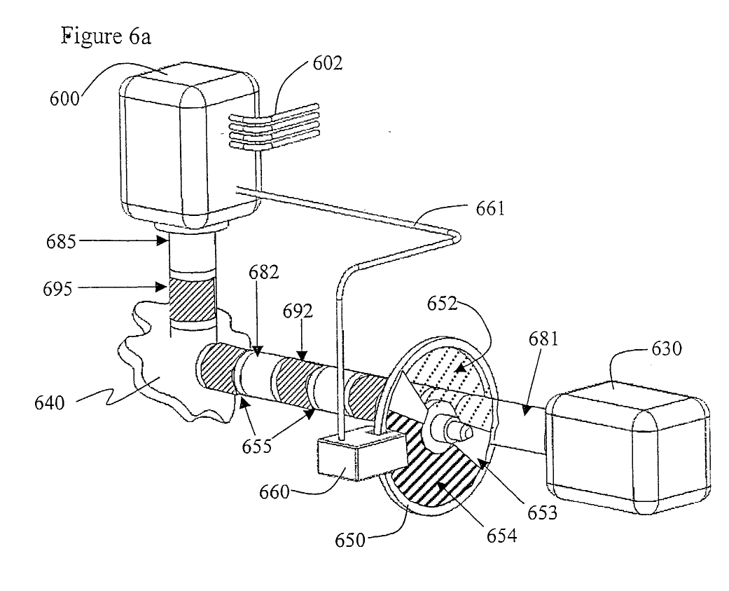

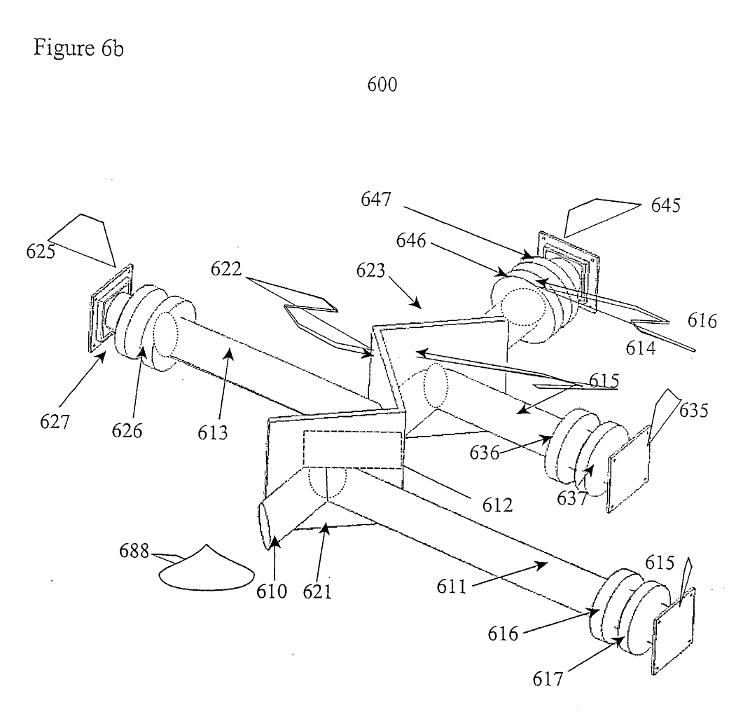

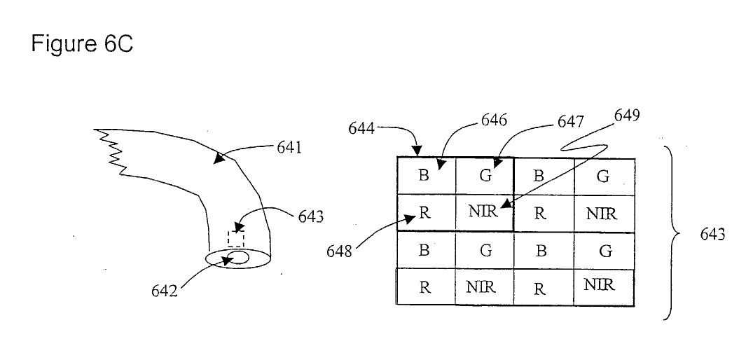

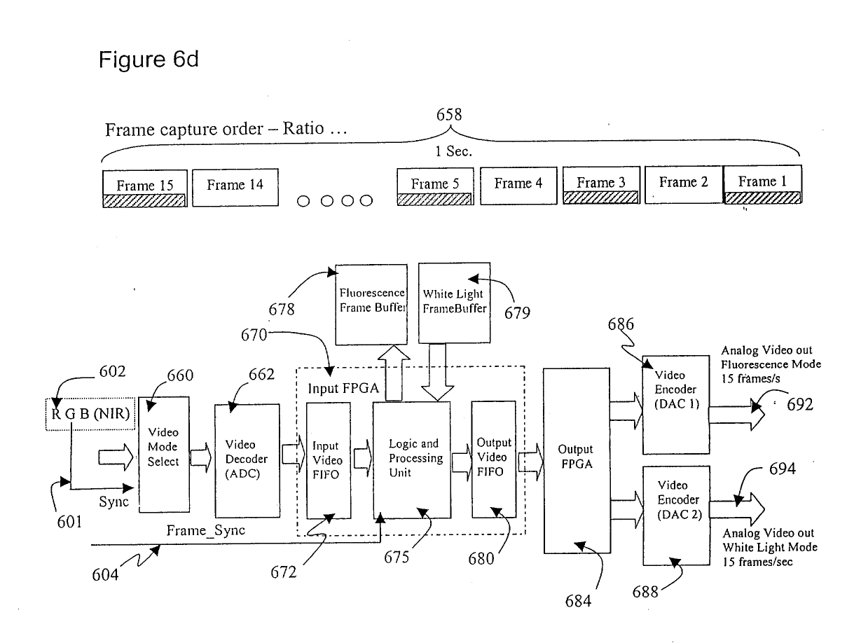

FIG. 4 shows a means of providing and modulating illumination for contemporaneous white light and fluorescence endoscopy for exploitation by the present invention. Endoscope 400 is provided with one or more illumination sources at the distal end 410. One advantage of such a configuration is that it eliminates transmission losses associated with the endoscope, which for certain wavelengths may be substantial. In addition, the fast switching of these devices provides a simple means to modulate the desired illumination(s). As depicted, three LEDs provide illumination and via electrical connections, may be synchronized for illumination and image detection. LED 451 for example, could provide a broad spectrum such as Spectrum 0 as discussed in association with FIG. 1. Typically this broad spectrum would be further modulated as will be discussed in association with FIGS. 5 and 6. LED 451 could also provide a narrower spectrum such as Spectrum 1 as discussed with FIG. 1. A second LED 452 could be provided with output such as Spectrum 3 or Spectrum 5 (as per FIG. 1) thereby supporting simultaneous white light and fluorescence endoscopy. Similarly, a third LED 453 having an illumination such as Spectrum 7 (as per FIG. 1) could extend imaging into the red and near-IR wavelength ranges. Various imaging modes and synchronization requirements will now be further described.

FIG. 5 illustrates an embodiment of the present invention providing simultaneous white light and fluorescence imaging. Light source 580 delivers broadband illumination (such as Spectrum 0 discussed in association with FIG. 1). The light source may be a single unit or be comprised of a combination of light sources to deliver the desired illumination. New higher powered LEDs provide useful spectra at intensity levels appropriate for use at the tip of an endoscope as described, or as part of the light source, for example blue LEDs of over 200 mW.

Accordingly, these light sources may be electronically switched at high rates (under 1 μsec) to provide modulation illumination spectra as described.

The emerging light beam 581 interacts with an optical modulator, which in this instance is rotating filter wheel 550, which consists of a white light or color balance filter 552 to provide an output spectrum (such as Spectrum 1 discussed in association with FIG. 1) for white light imaging, and a fluorescence excitation filter 554 to provide excitation light spectrum (such as Spectra 3, 5, or 9 as discussed in association with FIG. 1) for fluorescence imaging. The two optical filters 552 and 554 may further include a light blocking strip 553 to separate the spectral beams. Accordingly, light beam 581 is modulated into white light illumination segments 582 and fluorescence excitation segments 592 which may be spaced by unlighted segments 555. The modulated light beam contacts and interacts with a target object such as tissue 540 which may produce reflected white light segments 583 (with spectral content such as Spectrum 2 discussed in association with FIG. 1) and fluorescence emission segments such as 593 (with spectral components such as Spectra 4,6, or 10 discussed in association with FIG. 1). The imaging beam of spaced, alternative segments is then further processed by optical modulator 520, which in this instance is a second rotating filter wheel positioned at 45 degrees to the incident light generating imaging segments, 90 degrees apart from each other. The second optical modulator in this instance consists of an opening or a color balance filter 522 to pass the white light imaging segments 585, and filter 524, which could be a reflection mirror (approximating 100 percent reflectivity) to direct fluorescence imaging beam segments 595. The white light imaging segments arrive at detector 500 which could be an RGB video color camera outputting standard RGB and synchronization video signals 502 for processing and/or display. The fluorescence imaging segments arrive at detector 530 which could be a fluorescence imaging camera, outputting standard RGB and synchronization video signals 532, again for further processing and/or display.

Optical encoders 510, 560, function as frame sensors associated with optical modulators (rotating filter wheels) 550 and 520, respectively, and interface with synchronization device 570 via cables 571 and 572 to provide means to coordinate and synchronize the two optical modulators along with providing frame sync signals to control and synchronize white light detector 500 and fluorescence detector 530 via cables 574 and 573.

White light images from detector 500 and fluorescence images from detector 530 may be displayed on separate monitors or on different partitions of the same viewing monitor to be viewed simultaneously. Alternatively, because the two images are synchronized, they may be overlaid, processed, pseudo-colored or combined as required or desired.

Another useful image display mode would be to display the R (red) channel of the fluorescence imaging mode (alone or in combination with other display modes) as this R signal is generated by the near infrared reflectance signal 110R2 (Spectrum 10 of FIG. 1) which is less affected by blood absorption and thus may permit the physician to observe tissue structures through blood, for example to verify that a biopsy was performed at the desired location.

Various options such as spatial light modulators (SLMs) comprised of liquid crystals, digital micro-mirror devices (DMD), or other optical/electrical apparati incorporating gratings, prisms etc., may accomplish the same ends as the optical modulators discussed above. In general, solid-state devices with no moving parts may improve use factors such as reliability, and under electronic control may also simplify design by eliminating components such as the associated optical encoders.

In the illustrated embodiment, white light and fluorescence are having approximately a 50 percent duty cycle. Various other ratios, such as 25 percent for white light and 75 percent for fluorescence may be implemented as required or desired by changing the filter area or timing if another form of optical modulator is utilized.

FIG. 6a shows another embodiment of the present invention which reduces the number of components required to realize simultaneous multi-mode imaging. Illumination source 630 provides the broad-band illumination (such as Spectrum 0 discussed in association with FIG. 1). The emerging illumination 681 is further processed by optical modulator 650 which in this instance is a rotating filter wheel comprised of a white light or color balance filter 652 which passes modulated white illumination (such as Spectrum 1 discussed in association with FIG.

1) and fluorescence imaging filter 654 (which provides illumination such as spectra 3, 5, and 9 as discussed in association with FIG. 1). Filter wheel 650 may also utilize beam blocker 653. Accordingly, interleaved white light and fluorescence illumination segments such as 682 and 692 are produced with unlighted spacing segments 655, if desired. Illumination segments interact with a target object such as tissue 640. Reflected white light imaging segments such as 685 (with corresponding properties such as Spectrum 2 discussed in association with FIG. 1) and fluorescence imaging segments (with components such as those of Spectra 4, 6, 10 discussed in association with FIG. 1) are directed to detector 600. Frame sensor (optical encoder) 660 generates Frame_Sync signals as a means to indicate the position of the filter wheel 650, with synchronization information interfaced to detector 600 via communication cable 661. For example, a negative pulse on the Frame_Sync signal could be used to indicate timing for fluorescence detection while a positive pulse may indicate white light synchronization information. A detector 600 (detailed in FIG. 6b) receives the imaging segments and generates fluorescence imaging signal and white light imaging signal simultaneously via image processing electronics (shown and discussed with FIG. 6d). In a simple configuration, filter wheel 650 consists of two equal proportion filters 652 and 654 for white light illumination and fluorescence excitation, respectively. The wheel 650 rotates at 900 rpm or 15 rotations per second providing for 15 frames/second each for white light and fluorescence detection at similar light sensitivity. The filter areas may be provided in another ratio, for example to increase fluorescence sensitivity, which is typically lower than the intensity of reflected white light. U.S. patent application Ser. No. 09/741,731 by Zeng, entitled “Methods and apparatus for fluorescence and reflectance imaging and spectroscopy and for contemporaneous measurements of electromagnetic radiation with multiple measuring devices” (and continuation filing No. 10/028,568, Publication No. 2002/0103439) discusses these principals and is therefore included herein by reference.

FIG. 6b shows a detector configuration for multimodal contemporaneous acquisition of white light reflectance and fluorescence emission imaging utilizing a detector with multiple sensors (e.g. CCDs), thus reducing or eliminating mechanical switching mechanisms as used in prior art such as (368). Accordingly, detector 600 is comprised of at least three sensors such as sensor 615, sensor 625 and 645 which could be for blue, green and red light, for example. Typically it is advantageous to configure sensors with comparable path lengths, for example, from the surface of dichroic mirror 621, the distance to sensor 645 is substantially equivalent to the distance from that point to sensor 615. An additional sensor such as 635 may be provided for another imaging mode such as near-IR imaging.

Alternating imaging light segments 610 enter the detector 600 in the direction indicated by arrow 688. When a fluorescence imaging segment (such as 695, discussed in association with FIG. 6a) enters the detector (typical examples are spectra 104E, 106E or 110E and 110R2 as discussed in association with FIG. 1), some of this light 610 interacts (passes through) dichroic mirror 621, which has a cut-off wavelength of approximately 500 nm, for example, reflecting light below 500 nm (611) and transmitting light above 500 nm (612). The imaging segment then further interacts with dichroic mirror 622 having a cut-off wavelength around 600 nm, reflecting fluorescence components 613 in the 500 run to 600 nm towards sensor 625 (for green light), while transmitting imaging spectral components 614. Similarly, dichroic mirror 623 (optional with fourth sensor 645) divides the now substantially red spectral components into red and near infrared. This reflected fluorescence component 655 is further optically processed with band pass filter 636 (e.g. having out of band rejection>O.D. 5) and then focused by lens 637 to form an image on sensor 635. The transmitted reference imaging spectral component 656 is further filtered by band pass filter 646 (e.g. having out of band rejection>O.D. 5) which is then focused by lens 647 to form an image on sensor 645. These multispectral images and signals as well as synchronization signals are fed to the electronics (discussed with FIG. 6d) for further processing, control, and display.

Similarly, when a white light imaging segment, such as 685 discussed in FIG. 6a, enters the detector, its blue spectral component in the 400 nm to 500 nm range is reflected by dichroic mirror 621, this light 611 is then filtered by band pass filter 616, and then focused by lens 617 to form the blue image on blue CCD sensor 615. The green (500-600 nm) and red (600-700 nm) spectral components 612 transmit through dichroic mirror 621 and are incident on dichroic mirror 622, which reflects the green spectral components 613 onto band pass filter 626 and this light is then focused by lens 627 to form the green image on the sensor 625, while red spectral components to pass through the dichroic mirrors and are filtered and focused to form the red image(s) on the red sensor 645, and, if provided, the near-IR components to sensor 635. These multispectral images (R, G, B and perhaps near-IR) as well as synchronization signals are fed to the electronics discussed in FIG. 6d for further processing and generating standard video signal outputs for display and/or analysis.

Alternatively, if a near-IR image is desired (in additional to the red image) the dichroic mirror may be selected to pass the near-IR and reflect red light thus changing the position where these two images are sensed.

The gain and/or shuttle speed of each sensor will be changed between different imaging modalities to assure the optimal signal output for all imaging modalities which could have quite different optical signal intensities. While these gains and/or shuttle speeds vary dynamically, there are always fixed amplification relationships between different sensors and that relationship is different for different imaging modalities.

The multimodal images are viewed on any type of video image display device(s), such as a standard CRT monitor, an LCD flat panel display, or a projector. Because the images are available contemporaneously, but in multiple bands, the user can display the images in any variety of formats: The user can mix and match white, red, green, and blue color images separately or together with fluorescence, infrared, and near infrared images, separately or together, on the same or separate monitors.

FIG. 6c shows a different detector configuration for multimodal contemporaneous acquisition of white light reflectance, NIR reflectance, and fluorescence emission imaging utilizing a miniaturized single CCD sensor with patterned filter coating at the distal tip of an endoscope. A microlens 642 focuses the image onto CCD sensor 643, both mounted at the distal end of endoscope 641, which has either illumination fiber bundle to conduct illumination from a outside light source to illuminate the tissue or LEDs located at the same distal tip to provide tissue illumination. The different adjacent pixels on CCD sensor 643 are designed to capture images at different spectral bands, for example, pixel 646 (B) is designated to capture image in the blue band with corresponding high quality band pass filter coating to pass only light from 400 nm to 500 nm; pixel 647 (G) captures image in the green band with corresponding high quality band pass filter coating to pass only light from 500 nm to 600 nm; pixel 648 (R) captures image in the red band with corresponding high quality band pass filter coating to pass only light from 600 nm to 700 nm; while pixel 649 (NIR) captures image in the NIR band with corresponding high quality band pass filter coating to pass only light from 700 nm to 900 nm. This CCD sensor output R, G, B, NIR signals as well as synchronization signals similar to camera 600 as shown in FIG. 6b and these signals are fed to the electronics discussed in FIG. 6d for further processing and generating standard video signal outputs for display and/or analysis.

FIG. 6d shows the block diagram for synchronization and control of imaging as described for FIGS. 6a and 6b to realize simultaneous white light and fluorescence imaging. Imaging signals 602 from detector 600 provide alternating fluorescence and white light images (frames) into the Video Mode Select switch 660, which assigns these signals to independent analog to digital converters (ADCs) in Video Decoder 662 to digitize images. Video synchronization is provided in this instance by the green channel 601. Digitized images are fed to Input FPGA (field programmable gate array) 670 for processing. Inside the Input FPGA 670, the digitized images are directed to Input FIFO (first in first out) video buffer 672 and then into the programmable processing unit 675 which splits the images into white light imaging frames and fluorescence frames as determined by the Frame_Sync signal 604 connected to the processing unit 675. Two memory buffers communicate with FPGA 670: Frame Buffer 678 for temporary fluorescence image storage and Frame Buffer 679 for temporary white light image storage.

Various imaging processing functions may be implemented within FPGA 670, for example, x-y pixel shifting for R, G, and B images for alignment and registration. X-y pixel shifting means to shift the digital image (image frame) in the horizontal direction (x) and/or vertical direction (y), one or more pixels. Such processing eliminates the need for more complicated or mechanical mechanisms, thus simplifying alignment of sensors such as 615, 625, 635 and 645 discussed with FIG. 6b. Another programmable image processing function may take ratios of corresponding pixels in two or more images. The processed digital images are output by video FIFO 680 to the Output FPGA 684, which splits the fluorescence image frames and white light image frames into video encoder (DAC 1) 686 and video encoder (DAC 2) 688 respectively. Video encoders 686 and 688 with digital to analog converters (DAC) to transform the digital image signals, for example, to standard analog video signals 692 and 694 to be displayed on standard analog video monitors. In addition to providing for synchronization of optical modulation, the Frame Sync signal 604 may be utilized by the detector, for example as a means to switch between fixed gain settings employed by different imaging modalities.

In the embodiment described with FIGS. 6a, 6b, 6c and 6d, 15 frames/second of digital fluorescence images and 15 frames/second of digital white light images are generated to preserve the same light sensitivity (for fluorescence mode) as if the camera shown in FIG. 6b is acquiring fluorescence images and white light images in sequential (a imaging modality as outlined in U.S. application Ser. No. 09/741,731 by Zeng et al. titled “Methods and apparatus for Fluorescence and Reflectance imaging and spectroscopy and for contemporaneous measurements of electromagnetic radiation with multiple measuring devices”, along with continuation application Ser. No. 10/028,568, U.S. Publication No. 2002/0103439). The video encoders 686 and 688 still output standard video signals, i.e., 30 frames/second by repeating (duplicating) each of the 15 frames digital images once per second. If a higher frame rate, for example 30 frames/second digital fluorescence images and white light images are desired (proportionately decreasing the light sensitivity), this may be realized by rotating the filter wheel 650 (discussed with FIG. 6a) at the appropriate rate, in this instance, 1800 rpm (30 rotations per second).

While preferred embodiments of the present invention have been shown and described, it is envisioned that those skilled in the art may devise various modifications of the present invention without departing from the spirit and scope of the appended claims.

We claim:

1. An optical apparatus for simultaneous measurement of images, comprising
a light source delivering interrogating broadband radiation,
a first optical modulator separating said interrogating broadband radiation into a plurality of interrogating spectral segments corresponding to a plurality of imaging modalities,
a target object to interact with said interrogating spectral segments to produce returning radiation for imaging,
a second optical modulator separating said returning radiation from the target object into a plurality of returning spectral segments corresponding to said plurality of imaging modalities, and
at least one detector to receive said returning spectral segments and to process said returning spectral segments into images.

2. The apparatus of claim 1, further comprising means to synchronize said first optical modulator and said second optical modulator.

3. The apparatus of claim 1, further comprising means to display said images.

4. The apparatus of claim 3, wherein said means to display includes means to align said images.

5. The apparatus of claim 4, wherein said means to align comprises x-y pixel shifting.

6. The apparatus of claim 1, further comprising means to synchronize said first optical modulator and said second optical modulator and means to display said images.

7. The apparatus of claim 1, wherein said light source comprises a plurality of light emitting diodes in an endoscope.

8. The apparatus of claim 7, wherein said first optical modulator separates said interrogating broadband radiation by synchronized electronic switching of said light emitting diodes.

9. The apparatus of claim 1, wherein said first optical modulator comprises a moving filter having a plurality of modulating components corresponding to said plurality of imaging modalities, said components each having a duty cycle consisting of a ratio of said broadband radiation separated by said component to said broadband radiation.

10. The apparatus of claim 9, wherein said moving filter comprises a filter wheel.

11. The apparatus of claim 10, wherein said filter wheel revolves at approximately 1800 rpm.

12. The apparatus of claim 10, wherein said plurality of modulating components comprises at least a color balance filter component and a fluorescent excitation filter component.

13. The apparatus of claim 12, wherein said duty cycle for said fluorescent excitation filter component is less than or equal to approximately 50 percent.

14. The apparatus of claim 12, wherein the duty cycle for said fluorescent excitation filter is greater than 50 percent.

15. The apparatus of claim 12, wherein said fluorescent excitation filter component corresponds to said interrogating spectral segments having a spectral range of approximately 400 to 450 nm.

16. The apparatus of claim 12, wherein said fluorescent excitation filter component corresponds to said interrogating spectral segments having a spectral range of approximately 700 to 750 nm.

17. The apparatus of claim 12, wherein said fluorescent excitation filter component corresponds to said interrogating spectral segments having spectral ranges of approximately 400 to 450 nm and 700 to 750 nm.

18. The apparatus of claim 10, wherein said filter wheel includes a light-blocking area.

19. The apparatus of claim 10, wherein said moving filter comprises at least a color balance filter and a fluorescence excitation filter.

20. The apparatus of claim 9, wherein said moving filter includes a light-blocking area.

21. The apparatus of claim 1, wherein said first optical modulator comprises an optical switching device.

22. The apparatus of claim 21, wherein said optical switching device comprises a spatial light modulator.

23. The apparatus of claim 22, wherein said spatial light modulator comprises a liquid crystal device.

24. The apparatus of claim 22, wherein said spatial light modulator comprises a digital micro-mirror device.

25. The apparatus of claim 1, further comprising at least at least a white light detector and a fluorescence detector.

26. The apparatus of claim 1, wherein said second optical modulator comprises a moving filter having a plurality of modulating components corresponding to said plurality of imaging modalities, said components having a duty cycle consisting of a ratio of said broadband radiation separated by said component to said broadband radiation.

27. The apparatus of claim 26, wherein said moving filter comprises a filter wheel.

28. The apparatus of claim 27, wherein said filter wheel revolves at approximately 1800 rpm.

29. The apparatus of claim 26, wherein said plurality of modulating components comprises at least a color balance filter component and a fluorescent reflection filter component.

30. The apparatus of claim 29, wherein said duty cycle for said fluorescent reflection filter component is less than or equal to approximately 50 percent.

31. The apparatus of claim 29, wherein the duty cycle for said fluorescent reflection filter is greater than 50 percent.

32. The apparatus of claim 29, wherein said fluorescent excitation filter component corresponds to said interrogating spectral segments having a spectral range of approximately 400 to 450 nm.

33. The apparatus of claim 29, wherein said fluorescent reflection filter component provides near 100 percent reflection in a spectral range of approximately 300 to 800 nm.

34. The apparatus of claim 29, wherein said fluorescent excitation filter component corresponds to said interrogating spectral segments having spectral ranges of approximately 400 to 450 nm and 700 to 750 nm.

35. The apparatus of claim 27, wherein said filter wheel includes a light-blocking area.

36. The apparatus of claim 26, wherein said moving filter includes a light-blocking area.

37. The apparatus of claim 1, wherein said second optical modulator comprises an optical switching device.

38. The apparatus of claim 37, wherein said optical switching device comprises a spatial light modulator.

39. The apparatus of claim 38, wherein said spatial light modulator comprises a liquid crystal device.

40. The apparatus of claim 38, wherein said spatial light modulator comprises a digital micro-mirror device.

41. The apparatus of claim 1, wherein said at least one detector comprises at least a spectrometer.

42. A method of simultaneously measuring images, comprising
producing interrogating broadband radiation,
separating said interrogating broadband radiation into a plurality of interrogating
spectral segments corresponding to a plurality of imaging modalities,
interacting said interrogating spectral segments with a target object to produce returning radiation,
separating said returning radiation into a plurality of returning spectral segments corresponding to said plurality of imaging modalities, and
processing said returning spectral segments into images.

43. The method of claim 42, further comprising synchronizing said step of separating said interrogating broadband radiation with said step of separating said returning radiation.

44. The method of claim 43, further comprising displaying said images.

45. The method of claim 42, further comprising displaying said images.

46. The method of claim 42, further comprising aligning said images.

47. The method of claim 46, further comprising x-y pixel shifting.

48. The method of claim 42, wherein said method is applied to observation of said target object through blood.

49. The method of claim 42, further comprising producing broadband radiation from light-emitting diodes.

50. An optical apparatus for simultaneous measurement of images, comprising
a light source delivering interrogating broadband radiation,
an optical modulator separating said interrogating broadband radiation into a plurality of interrogating spectral segments corresponding to a plurality of imaging modalities,
a target object to interact with said interrogating spectral segments to produce returning radiation, and
a detector to receive and process said returning radiation, comprising
means to separate said returning radiation into a plurality of returning spectral segments corresponding to said plurality of imaging modalities, and
means to process said returning spectral segments into images.

51. The apparatus of claim 50, further comprising means to synchronize said optical modulator and said detector.

52. The apparatus of claim 51, further comprising means to display said images.

53. The apparatus of claim 50, further comprising means to display said images.

54. The apparatus of claim 53, wherein said means to display includes means to align said images.

55. The apparatus of claim 54, wherein said means to align comprises x-y pixel shifting.

56. The apparatus of claim 50, wherein said light source comprises a plurality of light emitting diodes in an endoscope.

57. The apparatus of claim 56, wherein said optical modulator separates said interrogating broadband radiation by synchronized electronic switching of said light emitting diodes.

58. The apparatus of claim 50, wherein said optical modulator comprises a moving filter having a plurality of modulating components corresponding to said plurality of imaging modalities, said components having a duty cycle consisting of a ratio of said broadband radiation separated by said component to said broadband radiation.

59. The apparatus of claim 58, wherein said moving filter comprises a filter wheel.

60. The apparatus of claim 59, wherein said filter wheel revolves at approximately 1800 rpm.

61. The apparatus of claim 59, wherein said plurality of modulating components comprises at least a color balance filter component and a fluorescent excitation filter component.

62. The apparatus of claim 61, wherein said duty cycle for said fluorescent excitation filter component is less than or equal to approximately 50 percent.

63. The apparatus of claim 61, wherein the duty cycle for said fluorescent excitation filter is greater than 50 percent.

64. The apparatus of claim 61, wherein said fluorescent excitation filter component corresponds to said interrogating spectral segments having a spectral range of approximately 400 to 450 nmn.

65. The apparatus of claim 61, wherein said fluorescent excitation filter component corresponds to said interrogating spectral segments having a spectral range of approximately 700 to 750 nm.

66. The apparatus of claim 61, wherein said fluorescent excitation filter component corresponds to said interrogating spectral segments having spectral ranges of approximately 400 to 450 nm and 700 to 750 nm.

67. The apparatus of claim 59, wherein said filter wheel includes a light-blocking area.

68. The apparatus of claim 58, wherein said moving filter includes a light-blocking area.

69. The apparatus of claim 50, wherein said optical modulator comprises an optical switching device.

70. The apparatus of claim 69, wherein said optical switching device comprises a spatial light modulator.

71. The apparatus of claim 70, wherein said spatial light modulator comprises a liquid crystal device.

72. The apparatus of claim 70, wherein said spatial light modulator comprises a digital micro-mirror device.

73. The apparatus of claim 50, wherein said detector comprises at least a white light detector and a fluorescence detector.

74. The apparatus of claim 50 wherein said means to separate comprises a plurality of dichroic mirrors.

75. The apparatus of claim 74, further comprising a plurality of filters.

76. The apparatus of claim 75, wherein said plurality of filters comprise at least one band pass filter.

77. The apparatus of claim 75, wherein said plurality of filters comprise at least one long pass filter.

78. The apparatus of claim 75, wherein said plurality of filters comprise at least one band pass filter and at least one long pass filter.

79. The apparatus of claim 74, further comprising a plurality of lenses.

80. The apparatus of claim 79, wherein said plurality of lenses focus said plurality of returning, spectral segments onto a corresponding plurality of sensors.

81. The apparatus of claim 80, wherein said sensors have gain and said gain is adjustable based on said imaging modalities.

82. The apparatus of claim 80, wherein said plurality of sensors comprise CCDs.

83. The apparatus of claim 50, further comprising means to adjust intensity of said returning spectral segments.

84. The apparatus of claim 83, wherein said means to adjust is said optical modulator.

85. The apparatus of claim 58, further comprising means to adjust intensity of said returning spectral segments based on said duty cycle.

86. The apparatus of claim 50, further comprising a frame sensor to synchronize said optical modulator and said detector.

87. The apparatus of claim 50, wherein said means to process comprises
a switch to assign said returning spectral segments to one of a plurality of analog to digital converters, wherein said plurality of analog to digital recorders digitize said returning spectral segments, and
a gate array to process said digitized returning spectral segments into images.

88. The apparatus of claim 87, wherein said gate array aligns said images.

89. The apparatus of claim 88, wherein said gate array aligns said images by x-y pixel shifting.

90. The apparatus of claim 88, wherein said gate array aligns said images by measuring ratios of corresponding pixels in a plurality of said returning spectral segments.

91. A method of simultaneously measuring images, comprising
producing interrogating broadband radiation,
separating said interrogating broadband radiation into a plurality of interrogating spectral segments corresponding to a plurality of imaging modalities,
interacting said interrogating spectral segments with a target object to produce returning radiation, and
detecting said returning radiation, comprising
separating said returning radiation into a plurality of interrogating spectral segments corresponding to a plurality of imaging modalities, and
processing said returning spectral segments into images.

92. The method of claim 91, further comprising synchronizing said separating said interrogating broadband radiation step and said detecting step.

93. The method of claim 92, further comprising displaying said images.

94. The method of claim 91, further comprising displaying said images

95. The method of claim 91, further comprising aligning said images.

96. The method of claim 95, wherein said aligning step comprises x-y pixel shifting.

97. The method of claim 91, wherein said method is applied to observation of said target object through blood.

98. The method of claim 91, further comprising producing said broadband radiation by light emitting diodes.

99. The method of claim 98, further comprising producing said broadband radiation in an endoscope.

100. The apparatus of claim 99, further comprising separating said interrogating broadband radiation by synchronized electronic switching of said light emitting diodes.

101. The method of claim 100, wherein said method is applied to observation of said target object through blood.

102. An optical apparatus for simultaneous measurement of white light and fluorescence images, comprising
means for providing a desired illumination,
means for modulating said illumination at video-rate for real-time imaging,
means for producing images by interaction of said illumination with a target object,
means for separating said images at video-rate,
means for detecting said separated images,
means for processing said detected separated images,
means for controlling detection and processing of said separated images, and
means for displaying at least one of said processed images.

103. An endoscope for obtaining contemporaneous images, comprising
a probe having an inner end to be located within a body, and an outer end to extend outside said body,
a light source to produce interrogating broadband radiation,
an optical modulator separating said interrogating broadband radiation into a plurality of interrogating spectral segments corresponding to a plurality of imaging modalities,
a target object within said body to interact with said interrogating spectral segments to produce returning radiation for imaging,
a lens to focus said returning radiation on a CCD sensor,

104. The apparatus of claim 103, wherein said light source is connected to said outer end and further comprising an illumination fiber bundle conducting said broadband radiation from said light source.

105. The apparatus of claim 103, wherein said CCD sensor comprises at least one set of pixels, wherein each said pixel within each said at least one set is coated with one of a band pass filter passing blue light, a band pass filter passing green light, a band pass filter passing red light, and a band pass filter passing near infrared light.

106. The apparatus of claim 105, further comprising means for generating a blue channel image from said blue light, a green channel image from said green light, a red channel image from said red light, and a near infrared image from said near infrared light.

107. The apparatus of claim 106, further comprising means to display said channel images.

108. The apparatus of claim 107, wherein said CCD sensor comprises at least one set of pixels, wherein each said pixel within each said at least one set is coated with one of a band pass filter passing blue light, a band pass filter passing green light, and a band pass filter passing red light.

109. The apparatus of claim 108, further comprising means for generating a blue channel image from said blue light, a green channel image from said green light, and a red channel image from said red light.

110. The apparatus of claim 109, further comprising means to display said channel images.

111. The apparatus of claim 103, wherein said optical modulator comprises a moving filter having a plurality of modulating components corresponding to said plurality of imaging modalities, said components each having a duty cycle consisting of a ratio of said broadband radiation separated by said component to said broadband radiation.

112. The apparatus of claim 111, wherein said moving filter comprises a filter wheel.

113. The apparatus of claim 112, wherein said filter wheel revolves at approximately 1800 rpm.

114. The apparatus of claim 112, wherein said plurality of modulating components comprises at least a color balance filter component and a fluorescent excitation filter component.

115. The apparatus of claim 114, wherein said duty cycle for said fluorescent excitation filter component is less than or equal to approximately 50 percent.

116. The apparatus of claim 114, wherein the duty cycle for said fluorescent excitation filter is greater than 50 percent.

117. The apparatus of claim 114, wherein said fluorescent excitation filter component corresponds to said interrogating spectral segments having a spectral range of approximately 400 to 450 mm.

118. The apparatus of claim 114, wherein said fluorescent excitation filter component corresponds to said interrogating spectral segments having a spectral range of approximately 700 to 750 mm.

119. The apparatus of claim 114, wherein said fluorescent excitation filter component corresponds to said interrogating spectral segments having spectral ranges of approximately 400 to 450 nm and 700 to 750 mm.

120. The apparatus of claim 112, wherein said filter wheel includes a light-blocking area.

121. The apparatus of claim 111, wherein said moving filter comprises at least a color balance filter and a fluorescence excitation filter.

122. The apparatus of claim 111, wherein said moving filter includes a light-blocking area.

123. The apparatus of claim 103, wherein said optical modulator comprises an optical switching device.

124. The apparatus of claim 123, wherein said optical switching device comprises a spatial light modulator.

125. The apparatus of claim 124, wherein said spatial light modulator comprises a liquid crystal device.

126. The apparatus of claim 124, wherein said spatial light modulator comprises a digital micro-mirror device.

127. An endoscope for obtaining contemporaneous images, comprising
a probe having an inner end to be located within a body, and an outer end to extend outside said body,
a light source comprising a plurality of light-emitting diodes located as said inner end and producing interrogating radiation, wherein said light-emitting diodes are electronically switched to produce a plurality of interrogating spectral segments corresponding to a plurality of imaging modalities,
a target object within said body to interact with said interrogating spectral segments to produce returning radiation for imaging,
a lens to focus said returning radiation on a CCD sensor,
wherein said CCD sensor comprises at least one set of pixels and said set of pixels captures a plurality of spectral bands of said image.

128. The apparatus of claim 127, wherein each said set of pixels comprises
a pixel coated with a band pass filter passing blue light,
a pixel coated with a band pass filter passing green light,
a pixel coated with a band pass filter passing red light, and
a pixel coated with a band pass filter passing near infrared light.

129. The apparatus of claim 128, further comprising means for generating a blue channel images from said blue light, a green channel image from said green light, a red channel image from said red light, and a near infrared image from said near infrared light.

130. The apparatus of claim 129, further comprising means to display said channel images.

131. The apparatus of claim 127, wherein each said set of pixels comprises
a pixel coated with a band pass filter passing blue light,
a pixel coated with a band pass filter passing green light, and
a pixel coated with a band pass filter passing red light.

132. The apparatus of claim 131, further comprising means for generating a blue channel image from said blue light, a green channel image from said green light, and a red channel image from said red light.
